(19) United States
(12) Patent Application Publication
(10) Pub. No.: US 2024/0285535 A1
(43) Pub. Date: Aug. 29, 2024

(54) PRINTED COMPOSITION FOR BIOMEDICAL USES

(71) Applicant: President and Fellows of Harvard College, Cambridge, MA (US)

(72) Inventors: Daniele FORESTI, Somerville, MA (US); Armand KURUM, Cambridge, MA (US); Jennifer A. LEWIS, Cambridge, MA (US)

(21) Appl. No.: 18/289,777

(22) PCT Filed: May 13, 2022
(86) PCT No.: PCT/US22/29169

§ 371 Date: Nov. 7, 2023

Related U.S. Application Data
(60) Provisional application No. 63/188,659, filed on May 14, 2021.

(51) Int. Cl. A61K 9/16; A61K 38/38; B29C 64/112; B29C 64/209; B29K 105/00; B29K 105/16; B29K 105/24; B33Y 10/00; B33Y 30/00; B33Y 70/00; C07K 14/765; C07K 16/06; C07K 16/32; C07K 17/04

(52) U.S. Cl. CPC A61K 9/1652; A61K 38/385; B29C 64/112; B29C 64/209; B33Y 10/00; B33Y 30/00; B33Y 70/00; C07K 14/765; C07K 16/06; C07K 16/32; C07K 17/04; B29K 2105/0061; B29K 2105/0094; B29K 2105/16; B29K 2105/24; B29K 2105/251; B29K 2995/0056; B29K 2995/0094

(57) ABSTRACT

A printed composition for biomedical uses comprises a liquid droplet prior to crosslinking and a gelled particle after crosslinking, where the liquid droplet comprises a formulation including a hydrogel precursor and abiologic, and the gelled particle comprises a cross-linked hydrogel matrix with the biologic dispersed therein. The formulation has a viscosity in a range from about 100 mPa-s to about 500,000 mPa-s.

### RELATED APPLICATION

The present patent document claims the benefit of priority under 35 U.S.C. 119(e) to U.S. Provisional Patent Application No. 63/188,659, filed on May 14, 2021, and hereby incorporated by reference in its entirety.

### TECHNICAL FIELD

The present disclosure is related generally to microparticle production and more specifically to a printed composition for biomedical applications.

### BACKGROUND

Hydrogels have become essential tools in tissue engineering, regenerative medicine, and drug delivery owing to their high water-content and biocompatibility (REF). Hydrogel microparticles in particular are seeing increased interest as delivery vehicles of drugs and cells, and as building blocks of macroscale granular structures. Their multiscale properties from the nanoscale (mesh size, electrostatic interactions), to the microscale (particle size and mechanical properties), and macroscale (interparticle interactions) provide unprecedented freedom in the design of biomaterial-based approaches for biomedical applications. In addition, hydrogel microparticles can be easily injected through needles and catheters due to their micron size, making them highly suited to in vivo administration. The hydrogel microparticles can be loaded with a variety of fragile biologics, such as therapeutic proteins, for local delivery. The modularity and potential of hydrogel microparticle-based systems reside in the ability to tune their properties at the micron scale, i.e., at the microparticle scale. The modulation of these properties may require changing the material composition and concentration or varying the microparticle production parameters.

Unfortunately, conventional microparticle production technologies, such as spray-drying and emulsification, can handle only a limited range of materials and tend to generate microparticles with high polydispersity. Such production methods may rely on high shear stresses, which can be detrimental to hydrogel microparticles with fragile and expensive cargo. Moreover, emulsion-based approaches, including droplet-based microfluidics, can expose these cargos to hydrophobic carrier fluids that damage the molecules. Post-processing steps such as sieving (spray drying, bulk emulsification) or washing (emulsions-based methods) can be required and potentially lead to material waste. The loss of cargo during production is a prohibitive aspect when dealing with costly biologics. These limitations make current technologies ill-suited for the generation and encapsulation of concentrated therapeutic antibody formulations, underscoring the need for new manufacturing technologies.

### BRIEF SUMMARY

A printed composition for biomedical uses comprises a liquid droplet prior to crosslinking and a gelled particle after crosslinking, where the liquid droplet comprises a formulation including a hydrogel precursor and a biologic, and the gelled particle comprises a cross-linked hydrogel matrix with the biologic dispersed therein. The formulation has a viscosity in a range from about 100 mPa·s to about 500,000 mPa·s.

A method of acoustophoretically printing a composition includes: arranging a nozzle within a first fluid, the nozzle having a nozzle opening; generating an acoustic field in the first fluid by an oscillating emitter; driving a formulation comprising a hydrogel precursor and a biologic out of the nozzle so as to form a pendant droplet comprising the formulation at the nozzle opening; detaching the pendant droplet by acoustic forces from the acoustic field, the formulation thereby being released in the first fluid as a liquid droplet; and crosslinking the liquid droplet to form a gelled particle comprising a crosslinked hydrogel matrix with the biologic dispersed therein.

### BRIEF DESCRIPTION OF THE DRAWINGS

FIGS. 1A-1E illustrate how acoustophoretic printing may be a platform for microparticle production: In FIG. 1A, droplets are formed at a nozzle tip, and their detachment controlled by exerting acoustophoretic forces at the nozzle tip; in FIG. 1B, the generated droplets are collected in a bath; FIG. 1C shows particle size distribution for different acoustophoretic forces (d=65 μm); FIG. 1D shows viscosity vs. shear rate rheological curves of alginate at different concentrations; and FIG. 1E shows acoustophoretically printed alginate microparticles at 10 wt. % concentration.

FIGS. 2A-2F illustrate the flow rate independence of continuous mode: FIG. 2A shows alginate microparticle production for a variable flow rate constant, step, and ramp; FIGS. 2B and 2C reveal that monodispersity is preserved with a high particle quality; FIG. 2D shows, in a periodic dripping regime, the droplet diameter D at detachment slightly decreases with flow rate Q, with the strong decrease at the dripping-to-jetting transition (scale bar=1 mm); in FIG. 2E, droplet diameter is normalized to D₀ (gₐ=0 g), and an increase of acoustic force does not show significant change of droplet diameter as flow rate is varied; FIG. 2F shows droplet diameter change with flow rate, normalized to D at low flow rate (Q=5 μL/min), where the droplet diameter becomes independent of flow rate for high acoustophoretic forces (gₐ=49 g).

FIG. 3A shows a schematic of alginate-protein microparticle production using acoustophoretic printing and electrostatic interactions between negatively-charged alginate and positively-charge antibodies; FIG. 3B shows zeta potential values of alginate, bovine serum albumin (BSA), and IgG; FIG. 3C shows the viscosity of alginate (25 mg/mL) mixed with BSA (25 mg/mL) at various pH values; FIG. 3D shows encapsulation efficiency of BSA (25 mg/mL) in alginate (25 mg/mL) microparticles increases with decreasing pH values of the formulation and crosslinking bath; and FIG. 3E shows (left) normalized turbidity measurements of alginate (25 mg/mL) and IgG (25 mg/mL) at various pH values, and (right) normalized turbidity of alginate (25 mg/mL) and IgG (25 mg/mL) at pH 5.5 with various concentrations of sodium chloride (NaCl).

FIG. 4A shows an alginate droplet containing IgG can be crosslinked in a bath containing calcium chloride and chitosan, where chitosan forms a shell around the alginate microparticle, preventing IgG leakage; FIG. 4B shows encapsulation efficiency of IgG (25 mg/mL) in alginate microparticles (uncoated) or chitosan-coated microparticles (CHI-coated); FIG. 4C shows encapsulation efficiency of various concentrations of IgG (100 mg/mL, 150 mg/mL, 200 mg/mL) in alginate microparticles coated with chitosan; FIG. 4D shows cumulative release of IgG (25 mg/mL) from alginate (25 mg/mL) microparticles; FIG. 4E shows activity of trastuzumab mixed with IgG at a 1:200 molar ratio following release from alginate (25 mg/mL) microparticles.

FIGS. 5A-5C illustrate acoustophoretic constant mode: in FIG. 5A, when the acoustophoretic field is constant, droplet detachment occurs when acoustic and gravity forces counteract the capillary force; FIG. 5B shows droplet volume at detachment is independent of the flow rate; and FIG. 5C shows droplet detachment by varying gₐ.

FIGS. 6A-6H show various plots showing characteristics of bovine serum albumin (BSA) solutions.

### DETAILED DESCRIPTION

In this work, acoustophoretic printing is described as an alternative to the current state-of-the-art for hydrogel microparticle manufacturing technology, enabling the generation of microparticles composed of high concentrations of polymers and biological cargos. This microparticle manufacturing technology is characterized by the absence of both high shear forces and hydrophobic carrier fluids, which is believed to be essential for encapsulating high viscosity formulations of active proteins and other biologics. To demonstrate this capability, hydrophilic polymer microparticles including high drug loadings are prepared via acoustophoretic printing, enabling the demonstration of polymer-to-biological cargo ratios above 1:50, and overall biologic concentrations larger than 160 mg/mL.

An acoustophoretically printed composition suitable for subcutaneous or intravenous delivery of a therapeutic agent is described herein. The composition comprises a liquid droplet prior to crosslinking and a gelled particle after crosslinking, where the liquid droplet comprises a formulation including a hydrogel precursor and a biologic, and the gelled particle comprises a crosslinked hydrogel matrix with the biologic dispersed therein. The composition may alternatively be described as a gelled particle comprising a cross-linked hydrogel matrix with a biologic dispersed therein, where the gelled particle is obtained by crosslinking a liquid droplet comprising a formulation including a hydrogel precursor and the biologic. Notably, the formulation has a relatively high viscosity in a range from about 100 mPa·s to about 500,000 mPa·s. The viscosity may be at least about 200 mPa·s, at least about 500 mPa·s, or at least about 1000 mPa·s, and is typically about 400,000 mPa·s or less, or about 200,000 mPa·s or less.

The gelled particle may be delivered subcutaneously or intravenously into a human body. Also or alternatively, the delivery or administration of the gelled particle may include one or more of the following: uricular, buccal, conjunctival, cutaneous, dental, electro-osmotical, endocervical, endosinusial, endotracheal, enteral, epidural, extra amniotical, extracorporeal, infiltration, interstitial, intra-abdominal, intra-amniotical, intra-arterial, intra-articular, intrabiliary, intrabronchial, intrabursal, intracardial, intracartilaginous, intracaudal, intracavernous, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastrical, occlusive dressing technique, ophthalmical, oral, oropharyngeal, parenteral, percutaneous, periarticular, peridural, perineural, periodontal, rectal, inhalation, retrobulbar, soft tissue, subarachnoidial, subconjunctival, subcutaneous, sublingual, submucosal, topical, transdermal, transmucosal, transplacental, transtracheal, transtympanic, ureteral, urethral, and/or vaginal administration. The gelled particle may have an encapsulation efficiency of at least about 55%, or at least about 80%.

The crosslinked hydrogel matrix of the gelled particle may comprise alginate, agar, agarose, carboxymethylcellulose, carrageenan, chitosan, chondroitin sulfate, collagen, dextran, fibrin, gelatin, hyaluronate, hydroxyethylcellulose, xanthan, polylysine, poly(acrylic) acid, poly(ethylene glycol) and its derivatives, cellulose and its derivatives, poly(propylene glycol) and its derivatives, polylactide and its derivatives, poly(glycolic acid) and its derivatives, poly(propylene fumarate) and its derivatives, polycaprolactone and its derivatives, polyhydroxybutyrate and its derivatives, polyacrylates and derivatives, poly(vinylpyrrolidone) and derivatives, and/or poly(ethylenimine) and its derivatives. Consistent with this, the hydrogel precursor employed for the formulation may comprise an alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and its derivatives, a precursor for cellulose and its derivatives, a precursor for poly(propylene glycol) and its derivatives, a precursor for polylactide and its derivatives, a precursor for poly(glycolic acid) and its derivatives, a precursor for poly(propylene fumarate) and its derivatives, a precursor for polycaprolactone and its derivatives, a precursor for polyhydroxybutyrate and its derivatives, a precursor for polyacrylates and derivatives, a precursor for poly(vinylpyrrolidone) and derivatives, and/or a precursor for poly(ethylenimine) and its derivatives.

The biologic may be a protein, hormone, peptide, nucleic acid, mammalian cell, micro-organism, small molecule, bacteria, drug (e.g., an antibody-based drug, such as monoclonal antibodies, antibody-drug conjugates, bispecific antibodies), cytokine (e.g., interleukin, interferon, tumor necrosis factor, chemokine, transforming growth factor beta, growth factor), insulin, Botulinum toxin type A, Botulinum toxin type B, bovine serum albumin (BSA), human immunoglobulin G (IgG), Fc fusion protein, anticoagulant, blood factor, bone morphogenetic protein, engineered protein scaffold, enzyme, thrombolytic, and/or another biological substance.

Advantageously, the biologic is homogeneously dispersed in the cross-linked hydrogel matrix. The particle may comprise a hydrogel-to-biologic ratio in a range from about 1:1 to about 1:1000. The ratio may be at least about 1:10, at least about 1:20, at least about 1:50, or at least about 1:100, and/or the ratio may be no greater than about 1:1000, no greater than about 1:800, or no greater than about 1:500. In some examples, a shell may encapsulate the gelled particle. Typically, the shell comprises a biocompatible polymer which may also be a biocompatible cationic polymer. Exemplary biocompatible polymers include chitosan and its derivatives and/or cationic dextran and its derivatives, cationic cellulose and its derivatives, catonic gelatin and its derivatives, Poly(2-N,N-dimethylaminoethylmethacrylate) and its derivatives, poly-L-ysine and its derivatives, polyethyleneeimine and its derivatives, poly(amidoamine)s and its derivatives. Typically, the gelled particles have an average diameter in a range from about 10 microns to about 2 mm, and they may be monodisperse, as described below.

The formulation may include the hydrogel precursor at a concentration of at least about 20 mg/mL, at least about 50 mg/mL, at least about 100 mg/mL, or at least about 200 mg/mL, and/or as high as about 1000 mg/mL, as high as about 800 mg/mL, or as high as about 600 mg/mL. With acoustophoretic printing as described herein, monodisperse hydrogel microparticles of an unprecedented range of concentrations (e.g., 2.5-10% w/w) and viscosities (above 200-15,000 cP) can be produced on demand with potentially zero waste and independently of flow rate. The formulation may also or alternatively include the biologic at a concentration of at least about 20 mg/mL, at least about 50 mg/mL, at least about 100 mg/mL, or at least about 200 mg/mL, and/or as high as about 1000 mg/mL, as high as about 800 mg/mL, or as high as about 600 mg/mL. Preferably, the formulation has a pH below an isoelectric point of the biologic, although in some examples the formulation may have a pH above the isoelectric point.

To stabilize the biologic (e.g., protein), an excipient may be included in the formulation. For example, the excipient may including one or more of the following: a buffering agent, such as citrate, phosphate, acetate and/or histidine buffer; an amino acid, such as L-arginine hydrochloride and/or L-glutamic acid, antioxidant, such as ascorbic acid, methionine, and/or ethylenediaminetetraacetic acid (EDTA); a surfactant, such as Polysorbate 80, Polysorbate 20, Brij 30 and Brij 35 and Pluronic F127, a preservative such as benzyl alcohol, cresol, phenol, and/or chlorobutanol.

The formulation may also or alternatively include an adjuvant, which may be described as a compound that can trigger an immune reaction. Adjuvants may be beneficial for vaccine delivery. Suitable adjuvants may include one or more of the following: an aluminum salt, such as amorphous aluminum hydroxyphosphate sulfate (AAHS), aluminum hydroxide, aluminum phosphate, potassium aluminum sulfate, and/or cytosine phosphoguanine (CpG).

A method of acoustophoretically printing a composition entails arranging a nozzle within a first fluid, which is typically air. The nozzle has a nozzle opening, which may be placed in opposition to a substrate or liquid bath. An acoustic field is generated in the first fluid by an oscillating emitter, and a formulation comprising a hydrogel precursor and a biologic (e.g., as set forth above) may be forced out of the nozzle so as to form a pendant droplet including the formulation at the nozzle opening. The pendant droplet may be detached by acoustic forces from the acoustic field, such that the formulation is released in the first fluid as a liquid droplet, which undergoes crosslinking to form a gelled particle comprising a crosslinked hydrogel matrix with the biologic dispersed therein.

More specifically, the hydrogel precursor undergoes crosslinking to form the crosslinked hydrogel matrix. The crosslinking may be initiated by a crosslinking reagent, heat, irradiation, and/or a change in pH. The crosslinking may take place before or after the liquid droplet is deposited on a substrate or enters a liquid bath, which may comprise a crosslinking solution. In one example, the crosslinking may take place in the first fluid, which may be air (e.g., prior to or after reaching the substrate). For example, the crosslinking may effected by exposure to ultraviolet radiation or a crosslinking reagent before or after the liquid droplet reaches the substrate.

Also or alternatively, the crosslinking may take place in the liquid bath. The liquid bath may comprise a crosslinking reagent solution containing, in one example, calcium chloride (e.g., 0.1 wt. %) adjusted to a suitable pH, e.g., with sodium hydroxide or with a chitosan (e.g., 0.25 wt. %) and acetic acid mixture. The suitable pH may be below an isoelectric point of the biologic. As described above, the formulation including the hydrogel precursor and the biologic that undergoes acoustophoretic printing may also have a pH below the isoelectric point of the biologic. The formulation may include the hydrogel precursor at a concentration of at least about 20 mg/mL and/or as high as about 100 mg/mL. Also or alternatively, the formulation may include the biologic at a concentration of at least about 20 mg/mL and/or as high as about 200 mg/mL. The gelled particles may remain in the liquid bath for a time duration from about 30 min to about 90 min.

### Microparticle Size Control and Precursor Viscosity Range

In acoustophoretic printing, acoustic waves are exploited to generate a net force on a pendant drop. In particular, the nonlinear effect of the acoustic field—namely radiation pressure—is able to exert a surface force surface Fa at the droplet interface (typically in addition to the gravity force F_{g}) so to overcome the capillary force F_{c}. The equation can be written as:

$${F}_{c} = π σ d = {F}_{g} + {F}_{a} = V ρ ( g + {g}_{a} ) \qquad ( 1 )$$

where F_{c}=πσd is the capillary force for a given liquid with surface tension σ, that opposes both the gravity force F_{g}=⅙πD³ρg=Vρg, where D is the drop diameter, V is the drop volume, ρ is the fluid density, and g is gravitational acceleration. The acoustophoretic acceleration gₐ, embeds all the nonlinear effects of the acoustic field and its modeling into a single parameter, and it is measured in units of g=9.81 m·s⁻². The parameter gascales with the square of the acoustic pressure P, i.e., gₐ×P². P is usually controlled by controlling the voltage of the sound source. By increasing gₐ, one can linearly decrease the droplet volume at detachment V:

$$V = π d σ / ρ ( g + {g}_{a} ) \qquad ( 2 )$$

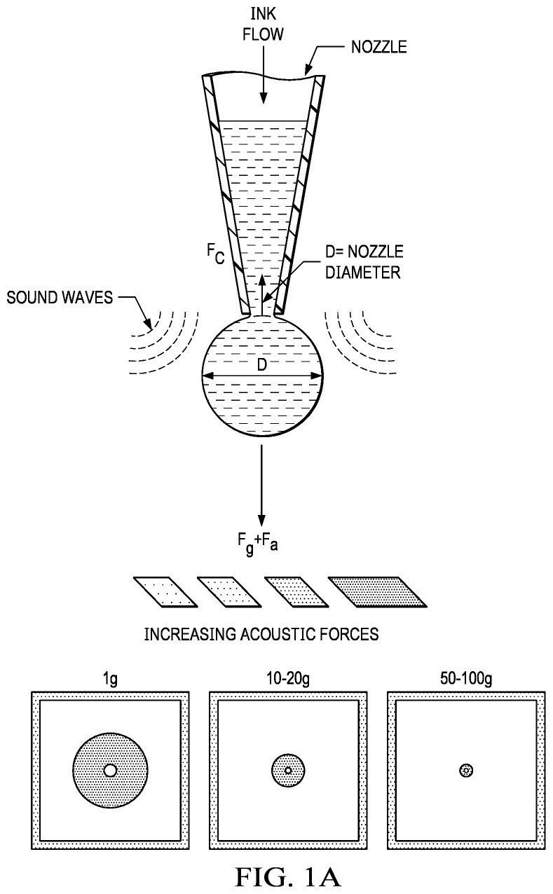

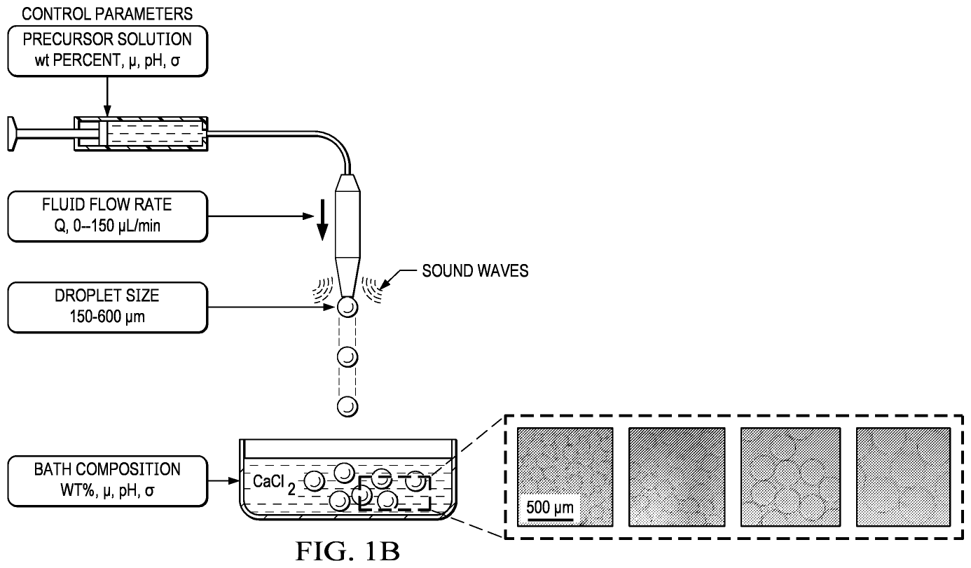

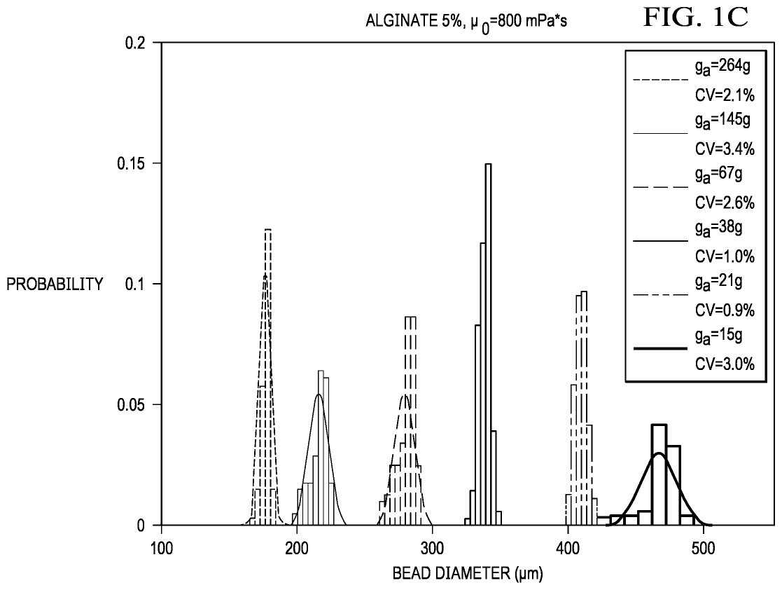

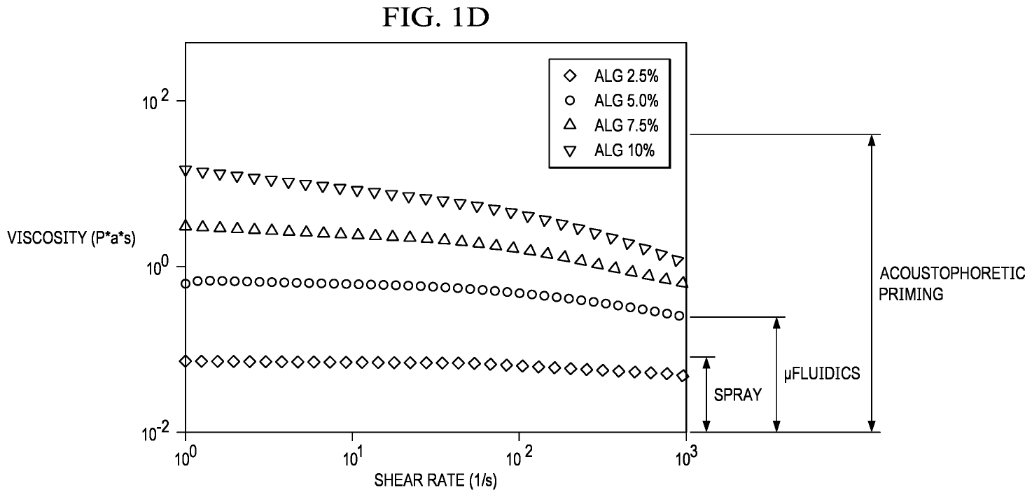

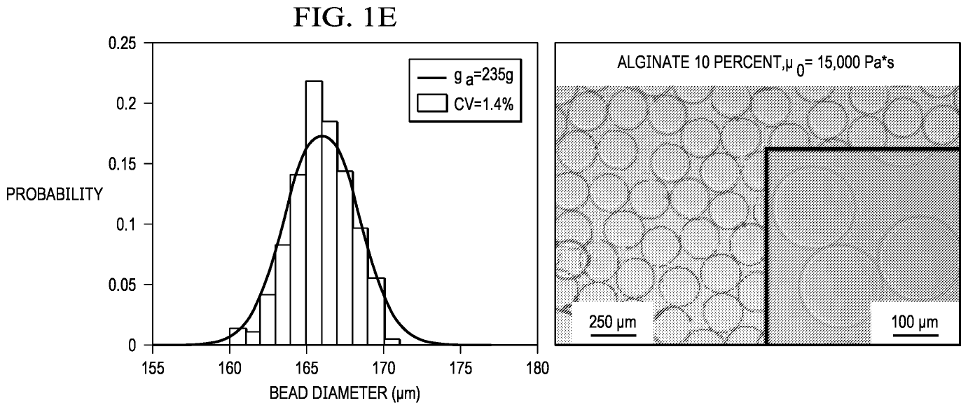

The formulation including the hydrogel precursor and the biologic may be flowed through a nozzle (typically having an outer diameter d=50-100 μm) and, in some examples, ejected into a crosslinking bath, as shown schematically in FIG. 1B. The crosslinking bath may include calcium chloride. The fluid flow rate may be constant or variable and may lie in a range from greater than 0 to 150 microliters per minute. The airborne nature of acoustophoretic printing makes it possible to vary independently different parameters to ensure the production of unique microparticles.

The acoustophoretic force allows for control over the microparticle size. FIG. 1C shows how acoustophoretic printing is able to produce monodisperse microparticles of different diameters (D=465±13 μm, 407±4 μm, 336±3 μm, 379±7 μm, 215±7 μm, and 176±4 μm) by only controlling the parameter gₐ (14.4 g, 21.4 g, 38.4 g, 67.2 g, 145.4 g, 264.6 g, respectively). Precise control over microparticle size distribution is beneficial for drug delivery kinetics and for good manufacturing practice requirements. Values of gₐ above 250 g are reported, which is about 2-fold increase over the inventors' previously published work, thanks to improvements in the acoustic field resonance. Despite the high viscosity of the 5 wt. % alginate solution (apparent viscosity μ₀=800 mPa·s), the monodispersity is conserved independently of the gₐ applied, with coefficient of variation (CV) below 4%.

The precursor composition has very few constraints (FIG. 1B, control parameter p and wt. %). Indeed, viscosity plays little or no role (Eq. 1). The ability of acoustophoretic printing to produce hydrogel microparticles at very high concentrations of alginate (10 wt. %) with viscosity μ₀≈15,000 mPa·s (FIG. 1D) is demonstrated. FIG. 1E shows the ability of acoustophoretic printing not only to successfully process 10 wt. % alginate microparticles, but also maintain a high level of monodispersity (CV=1.4%).

### Flow Rate Independence: On-Demand Microparticles

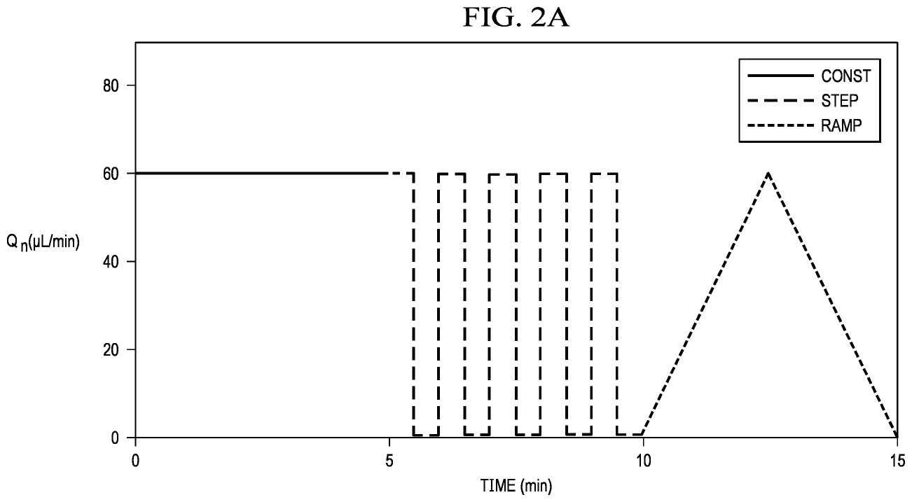

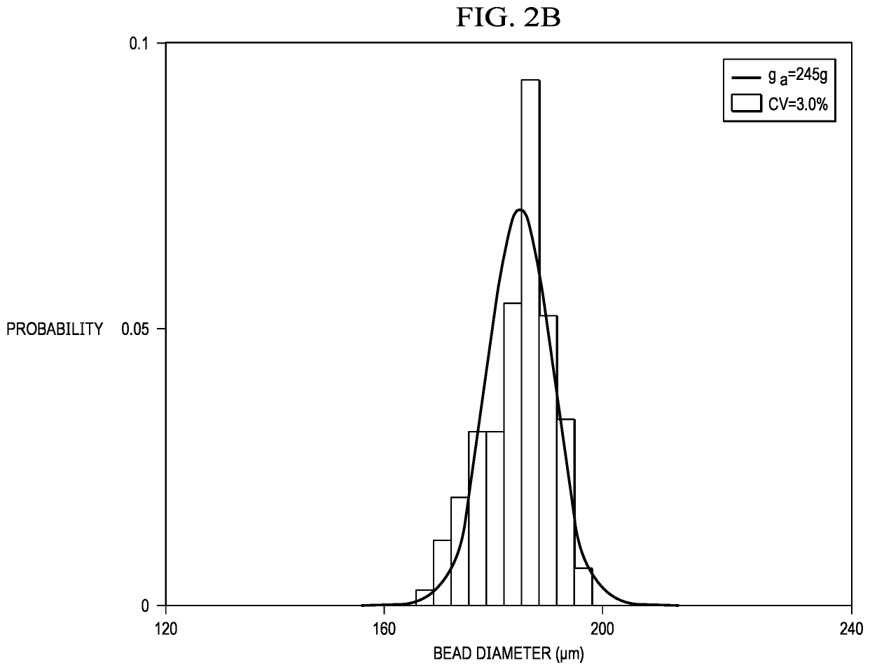

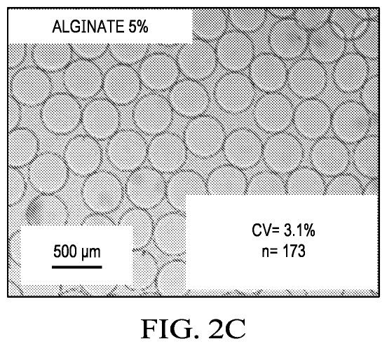

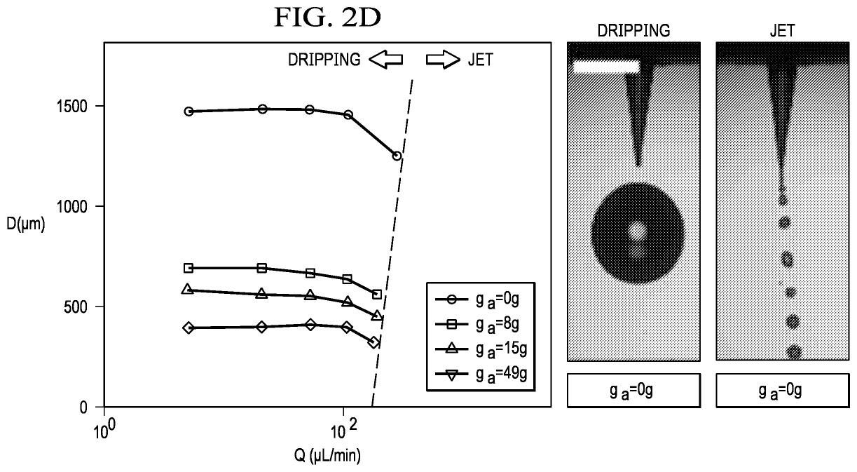

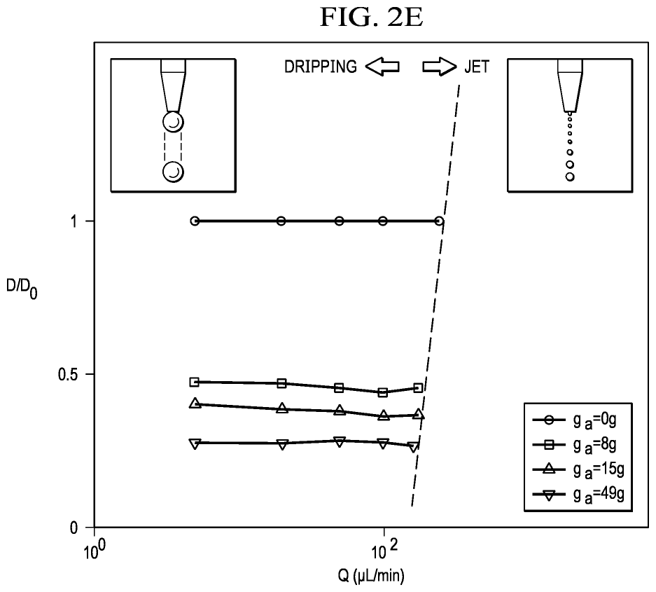

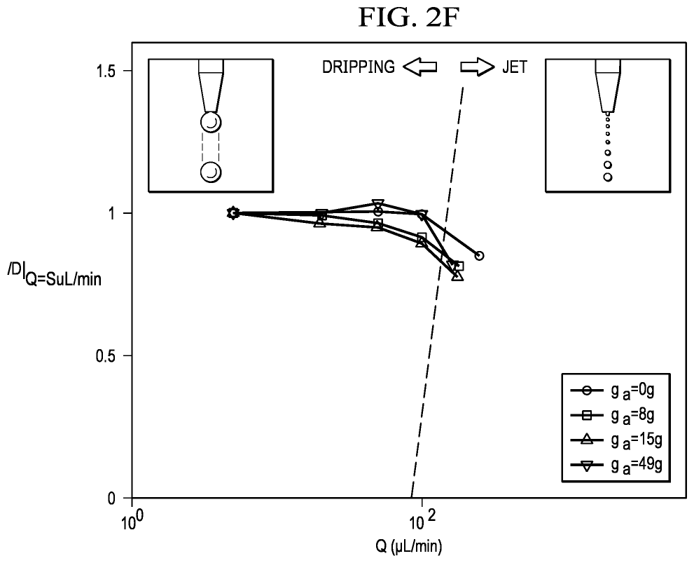

A key aspect of acoustophoretic printing is the decoupling between flow rate and droplet detachment (FIG. 1B, control parameter Q). Indeed, Equation 1 does not contain any information regarding the fluid flow rate Q. To demonstrate this, a syringe pump was used to control the nominal flow rate Qn. In FIG. 2A the flow rate is kept constant for the first five 5 minutes of ejection at 60 μL/min, followed by 5 minutes of a step function (from 60 μL/min to 0.60 μL/min every 30 seconds), to end with a ramp function (ramp up 2 minutes and 30 seconds till 60 μL/min, ramp-down to 0 μL/min in 2 minutes and 30 seconds. The microparticle size distribution is unaffected by the variation of flow rate (CV=3.1%, gₐ=245 g). This quasi drop-on-demand approach can be extremely convenient in microparticle production, making it a very robust process for microparticle production. Additionally, it eliminates the need for long ramping up time, reaching of equilibrium, and droplet formation—typically in the minutes range for microfluidics.

The flow rate dependence of ejected droplet diameter is investigated by using water as a model fluid. FIG. 2D shows a typical dripping to jetting transition of a pendant drop nozzle for low viscosity medium (water). Without applying any acoustic field (i.e. gₐ=0), the droplet size is fairly constant at D=1500 μm, decreasing sharply to 1200 μm just prior jetting regime transition. This behavior is consistent with the literature, especially since external disturbances (vibrations, surrounding air flow, etc.) can strongly influence the detachment. Similarly, higher acoustophoretic forces (gₐ=8, 15, and 49 g) induce the dripping-to-jetting transition at slightly smaller flow rate (240 μL/min for gₐ=0 to 150 μL/min for gₐ=49 g). For d=60 μm, flow rate above 150 μl/min can be achieved—about an order of magnitude higher than microfluidics devices.

To better analyze this behavior, FIG. 2E shows the droplet diameter D as normalized to the gₐ=0 droplet diameter D₀. At low values of gₐ, a slight decrease of droplet size with the increase of the flow rate is observed (within error). Since it is speculated that most of the variation is due to small disturbances, higher gₐ values should decrease this effect. In FIG. 2F the droplet diameter D is normalized to the smallest flow rate, Q=5 μl/min. By increasing the acoustophoretic forces, the flow rate dependence is significantly reduced up to the dripping-to-jetting transition. These results are consistent with the microparticle distribution results at high values of gₐ (gₐ=245 g, FIGS. 2B-2C), in which the diameter distribution seems very narrow independently of the flow rate Q for the entire regime of dripping regime.

### Protein Encapsulation in HMP

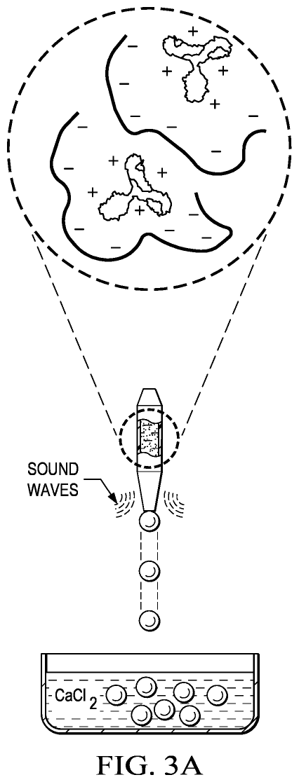

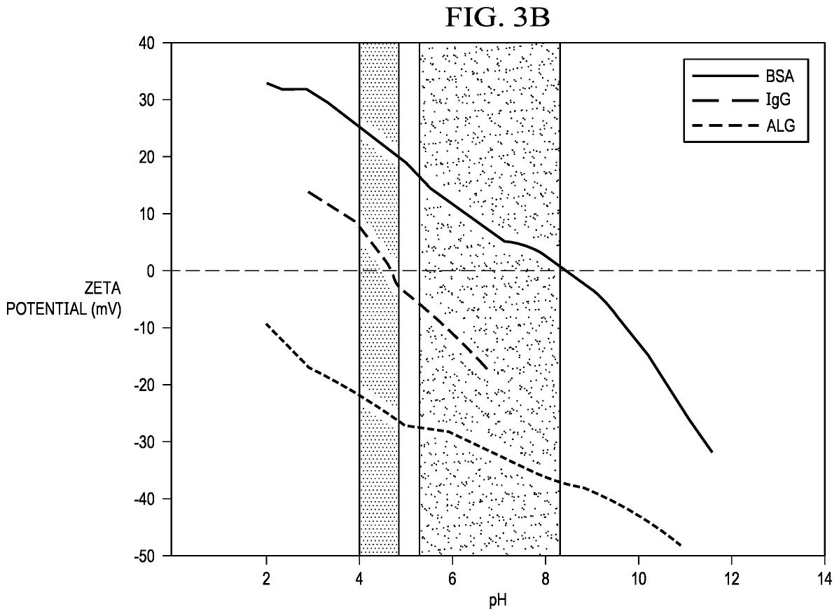

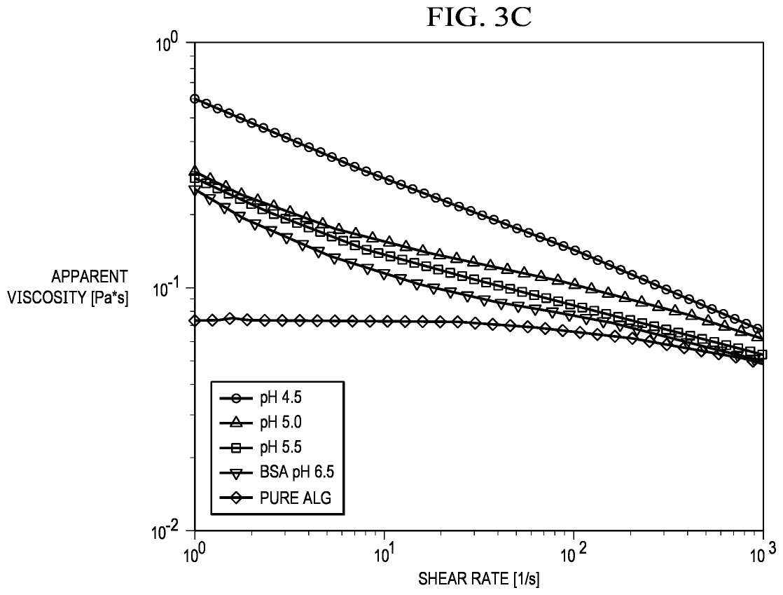

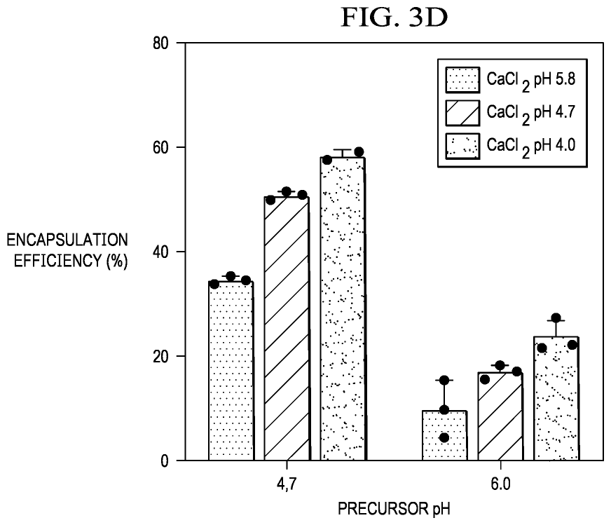

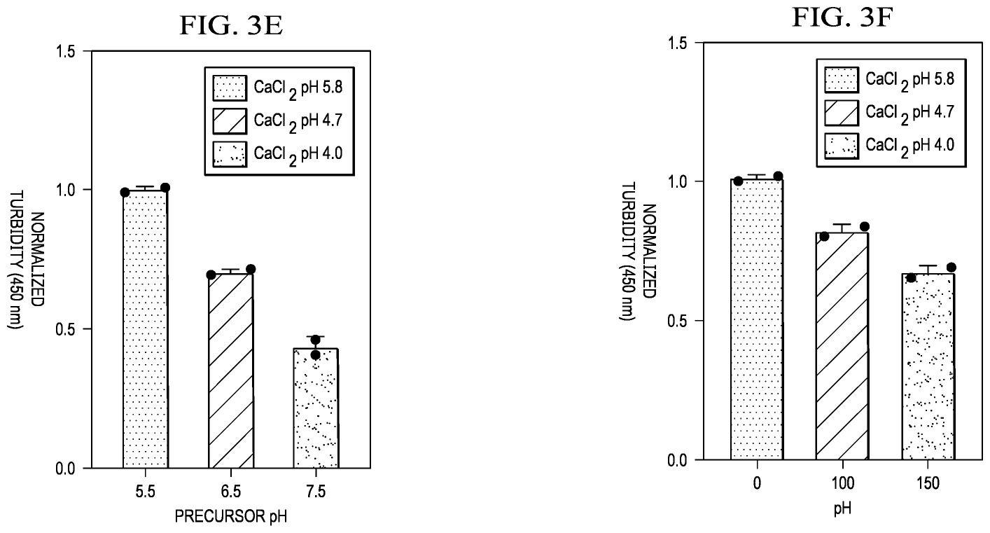

After the ability of acoustophoretic printing to produce microparticles of alginate at high concentration was explored, its potential in producing microparticles with high loading of proteins was investigated, using bovine serum albumin (BSA) and human immunoglobulin G (IgG) as model proteins. An objective was leveraging electrostatic interactions between negatively-charged alginate and positively-charged proteins to prevent protein diffusion from the polymer matrix (FIG. 3A). Owing to the overall negative charge of BSA at physiological pH, it is expected that the ionic interactions between alginate and BSA would become significant around the isoelectric point (pI) of BSA at pH 4.7 (FIG. 3B). Since the viscosity of a formulation is partially governed by the strength of the molecular interactions between alginate and protein in solution, the formulation rheology can be used as a measure of the complexation between the proteins and alginate. As expected, rheological data of alginate-BSA showed an increase in viscosity at pH values below the pI of BSA, consistent with expectations (FIG. 3C). Lowering the pH from 5.0 to 4.5 is sufficient to increase the formulation viscosity by a factor of 2.5 at low shear rates. Interestingly, a formulation composed of 25 mg/mL of BSA and 25 mg/mL of alginate is already too viscous for most conventional microparticles technologies (FIG. 3C, FIG. 1C). It was hypothesized that enhancing the interactions between BSA and alginate through modulation of pH value of the alginate-BSA formulation and the crosslinking bath could increase the encapsulation efficiency of BSA. In line with the hypothesis, varying the pH of the crosslinking bath from pH 5.8, above the pI of BSA, to pH 4.0 increased the retention of BSA inside the microparticle by a factor X. Similarly, changing the pH of alginate-BSA formulation from pH 6.0 to pH 4.0 increases encapsulation efficiency. IgG, a molecule with an overall positive charge at physiological pH (FIG. 3B) was also studied for its interactions with alginate. Turbidity measurements were used to assess the complexation at different pH values between alginate and IgG (FIG. 3F). The expected strength of the interactions correlate with an increase in turbidity with decreasing pH. Weak electrostatic interactions can be reversed at increasing pH or adding salts with shields the charges, providing as simple release mechanism at physiological conditions. Addition of sodium chloride at concentrations close to physiological conditions resulted in a decrease of the turbidity (FIG. 3F).

Encapsulation of Extreme Concentrations of IgB and mAbs in Core-Shell HMP

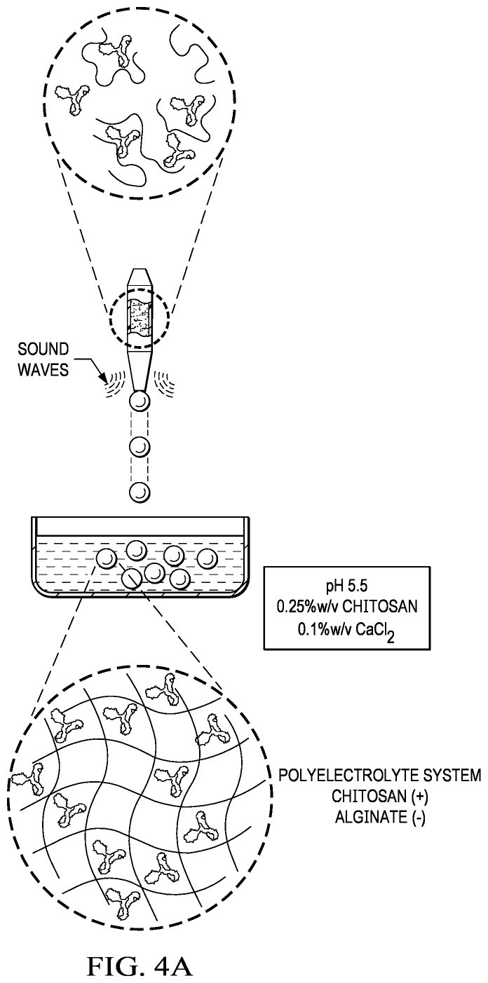

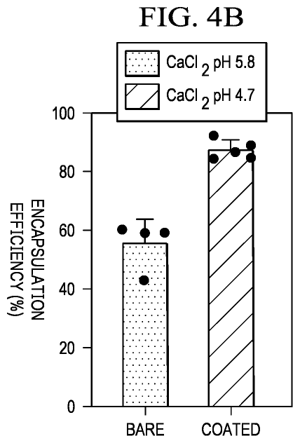

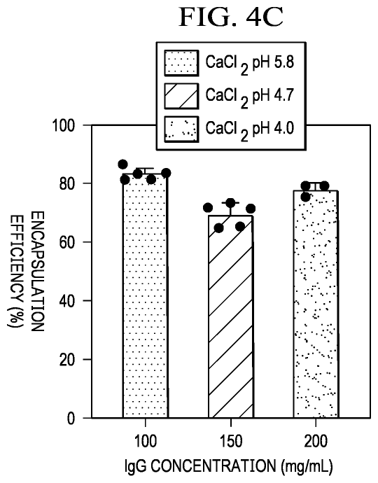

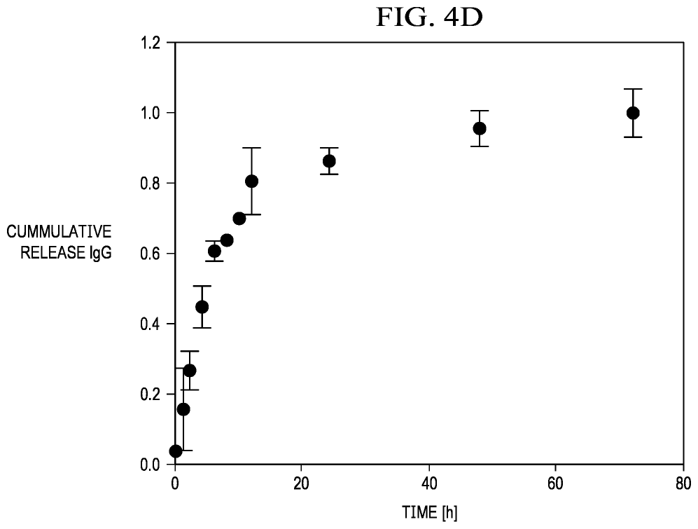

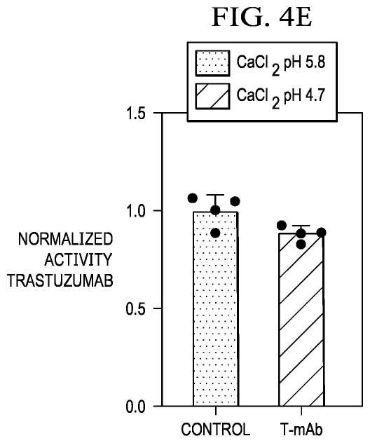

Using pH 5.5 for the formulation and crosslinking bath, IgG-alginate microparticles (FIG. 4A) produced at low calcium chloride concentration (0.1% wt) exhibit an encapsulation efficiency close to 60% (FIG. 4B). To further improve the encapsulation efficiency, a low concentration of chitosan (0.25% w/v), a biocompatible cationic polymer, was added to the crosslinking bath. Upon penetration of the IgG-alginate droplet into the crosslinking bath, a chitosan shell is formed around the IgG-alginate core due to electrostatic interactions between oppositely charged polymers. This facile strategy enables to reach encapsulation efficiencies above 80% (FIGS. 4A and 4B). Antibodies can be easily denatured by harsh processing conditions such as low pH or high shear stresses. The airborne nature of acoustophoretic printing means that droplet ejection generates very low stress to the encapsulated cargo, as it has shown to safely eject human stem cells. As a result, the activity of IgG was unchanged across various flow rates (2.5-50 μL/min) and acoustophoretic acceleration (0-100 g). Using the facile core-shell approach established at protein concentrations of 25 mg/mL, it was demonstrated that IgG concentrations up to 200 mg/mL can be ejected and encapsulated (FIG. 4C). Interestingly, at these IgG concentrations, droplet ejection failed after a few minutes. Upon investigating this issue, it was discovered that the droplet neck was drying during droplet formation, resulting in ejection failure. This phenomenon was observed previously at high polymer concentrations. In constant acoustophoretic field, the droplet is continuously pulled until it detaches, creating a region of enhanced evaporation. Varying the acoustic field over time (pulse mode) prevented this phenomenon. Crosslinked microparticles (100 mg/mL IgG) were subsequently resuspended in buffer mimicking physiological conditions in vitro to study the release of encapsulated IgG (FIG. 4D). As expected, the interactions between IgG and alginate were reversible at physiological conditions and the encapsulated protein was released very rapidly within the first 12 h (75% release) and then much slower over the next days. Finally, a pharmaceutically-relevant monoclonal antibody (Trastuzumab) was used to study whether its binding activity was preserved during manufacturing. IgG and trastuzumab were mixed at a molar ratio of 200:1 and encapsulated as shown previously. The activity of the trastuzumab released from the chitosan-coated alginate microparticles was preserved (FIG. 4E).

### Conclusions and Outlook

A novel microparticle production approach that enables processing of highly viscous formulations, including highly concentrated polymer and biologic (e.g., protein) formulations has been demonstrated. By decoupling droplet generation by fluid flow and crosslinking mechanism, monodisperse alginate microparticles up to 10 wt. % have been produced, independently of the flow rate. Formulations of 2.5 wt. % alginate containing up to 200 mg/mL of IgG may also be ejected, and high encapsulation efficiency (80%) is attained by using a simple core-shell approach. Owning those characteristics, acoustophoretic printing has a great potential to complement existing HMP manufacturing technologies.

### Materials and Methods

### Materials

Albumin Standard (Thermo Scientific, 23209), Alginic acid sodium salt (Sigma-Aldrich, 180947), Bovine Gamma Globulin Standard (Thermo Scientific, 23212), Bovine Serum Albumin (Proliant, 68700), Calcium chloride dihydrate (Sigma-Aldrich, 223506), Chitosan (Sigma-Aldrich, 448877), Slide-A-Lyzer MINI Dialysis (2 mL, 20K MWCO, 88405), Compat-Able Protein Assay (Thermo Scientific, 23215), Coomassie (Bradford) Assay Kit (Thermo Scientific, 23200), Human Total IgG Platinum ELISA (Thermo Scientific, BMS2091), Humanized Anti-HER2 ELISA Assay Kit (Eagle Bio, AHR31-K01), Immunoglobulin G (Equitech-Bio, SLH56), MES monohydrate (Sigma-Aldrich, 69889)

### Alginate—Immunoglobulin Formulation

Alginic acid sodium salt was dissolved in deionized water at a concentration of 150 mg/mL using a Speedmixer (Flacktek). The stock solution was stored at 4° C. Human immunoglobulin G (IgG) was dissolved in MQ water at a concentration varying between 130 mg/mL-200 mg/mL in deionized water. The IgG solution was centrifuged at 11,000 g for 15 min to remove insoluble aggregates. The solution was dialyzed against sodium acetate buffer (pH 5.5 30 mM) using a Slide-A-Lyzer (2 mL, 20 kDa cut-off) for 2 h a low shaking (100 rpm) at room temperature, the buffer was exchanged, and the dialysis tube placed at 4° C. for overnight dialysis. The solution was centrifuge at 11,000 g for 15 min to remove insoluble aggregates. The protein concentration was measured using Bradford assay and Gamma Globulin standards.

### Alginate—Immunoglobulin Microparticles Preparation

Alginate was adjusted to pH 5.0 (125 mg/mL) and gently mixed with a solution of IgG. Final concentration of alginate was typically 25 mg/mL and final concentration of IgG varied between 25 mg/mL-200 mg/mL. The formulation was adjusted to pH 5.5 by dropwise addition of acetate buffer (0.5M pH 5.5) if necessary. The formulation was centrifuged at 10,000 g for 15 min to remove any non-soluble aggregates. 1-2 mL of formulation were transferred to a plastic barrel (REF) or plastic syringe (REF) for acoustophoretic printing. A precise volume of alginate-IgG formulation (15 μL) was ejected through a glass-pulled nozzle (60-80 μm) at various equivalent acoustophoretic accelerations (TBD) in 24 well plate with 2 mL of crosslinking per well. The crosslinking buffer was either 0.1 wt % calcium chloride adjusted to pH 5.5 with sodium hydroxide (5N) or calcium chloride (0.1 wt %) with chitosan (0.25 wt %). Briefly, chitosan was stirred in 0.1 M acetic acid at 300 rpm at 60° C. for 6 h, 0.1% wt calcium chloride was added, and the final pH was adjusted to 5.5. The final buffer was mixed (Flacktek) at high speed and centrifuged at 5000 rpm to remove insoluble particles. Finally, 0.05% Tween 20 was added to the crosslinking buffer. During microparticle production, the crosslinking buffer was constantly stirred to avoid particles clumping. The particles were crosslinking for 1 h and collected from the crosslinking buffer.

### Encapsulation Efficacy

The protein concentration in the crosslinking bath was measured using a Bradford assay and a standard plate reader. The protein encapsulation efficiency was measured as follows

$$EE ( \% ) = \frac{{m}_{protein . buffer}}{{m}_{protein . total}} = \frac{{m}_{protein . buffer}}{Q \cdot {c}_{protein} \cdot {t}_{printing}} \qquad ( 3 )$$

$${m}_{protein . buffer} = mass of protein in crosslinking buffer [ mg ]$$

$${m}_{protein . total} = total ejected mass of protein [ mg ]$$

$$Q = ejection flow rate [ mL / min ]$$

$${c}_{protein} = protein concentration in formulation [ mg / mL ]$$

$${t}_{printing} = ejection time [ min ]$$

### Protein Activity

Microparticles were resuspended in 0.5-2 mL of 0.2M citrate buffer (pH 6.0) with 0.3 M sodium chloride and the solution was shaked until the microparticles were fully dissolved. The protein activity was measured using an enzyme-linked immunosorbent assay.

### Protein In Vitro Release

The microparticles (15 μL) were resuspended in HEPES-buffered Tyrode solution (2 mL) in a centrifuge tube. The microparticles were vigorously pipetted and placed in an incubator at 37° C. on orbital shaker at fixed speed (100 rpm). 200 μL of solution was collected at every time point for protein concentration measurements and 200 μL of fresh buffer was added. At the end of the experiment, the microparticles were dissolved in citrate buffer to quantify the amount of IgG left inside the microparticles.

### Rheology

Formulation rheology was characterized using a controlled-stress rheometer (Discovery Hybrid Rheometer 3; TA Instruments) equipped with 40 mm cone plate geometry (2:00:48 deg:min:sec) and a 250 μm gap.

### Alginate—Bovine Serum Albumin Formulation

Alginate was adjusted to the desired pH (100 mg/mL) and gently mixed with a solution of BSA. Final concentration of alginate was typically 25 mg/mL and final concentration of BSA varied between 25 mg/ml-200 mg/mL. The pH of the formulation was further adjusted if needed by dropwise addition of acetate buffer (0.5M) if necessary. 1-2 mL of formulation were transferred to a plastic barrel (REF) or plastic syringe (REF) for acoustophoretic printing. A precise volume of alginate-BSA formulation (15 μL) was ejected through a home glass-pulled nozzle (60-80 μm) at various equivalent acoustophoretic accelerations in 24 well plate with 2 mL of crosslinking per well. The crosslinking buffer was either 0.1% w/w calcium chloride adjusted to the desired pH with sodium hydroxide (5N). Finally, 0.05% Tween 20 was added to the crosslinking buffer. During microparticle production, the crosslinking buffer was constantly stirred to avoid particles clumping. The particles were crosslinking for 1 h and collected from the crosslinking buffer.

### Acoustophoretic Printing: Droplet Volume Evolution and Detachment in the Time Domain

The acoustophoretic droplet ejection process described in Eq. 1 refers to a quasi-static system. It is possible to extend the description to account for the evolution of the droplet size with respect to time, i.e. a dynamic model. By feeding the nozzle at a constant flowrate Q, the volume of the pendant drop would evolve as V(t)=Q·t, where t represents time. Equation 1 would become, in case of constant acoustic field, gₐ=const, and detachment would occur when:

$$V ( t ) ρ ( g + g a ) = Q \cdot t \cdot ρ ( g + ga ) = Fc = πσ d \qquad ( 4 )$$

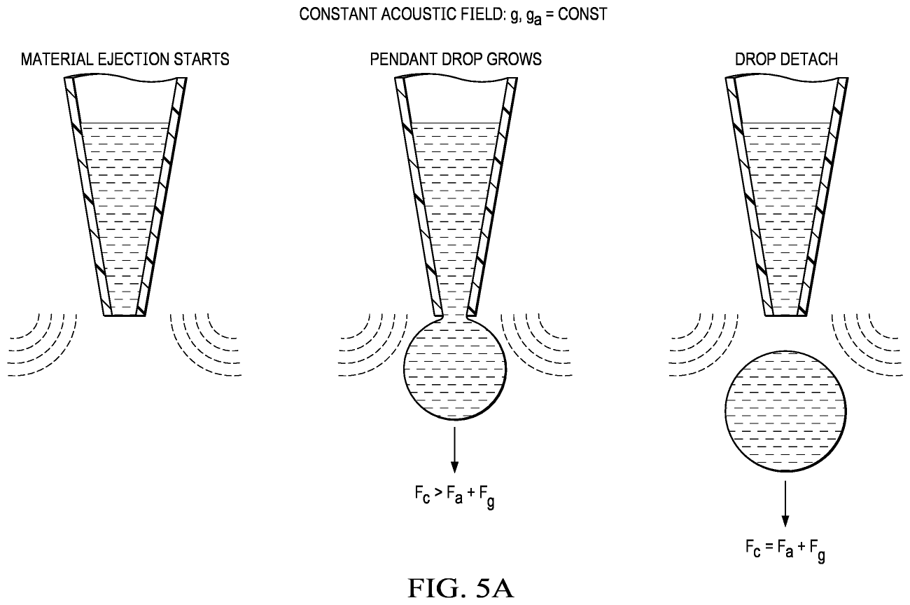

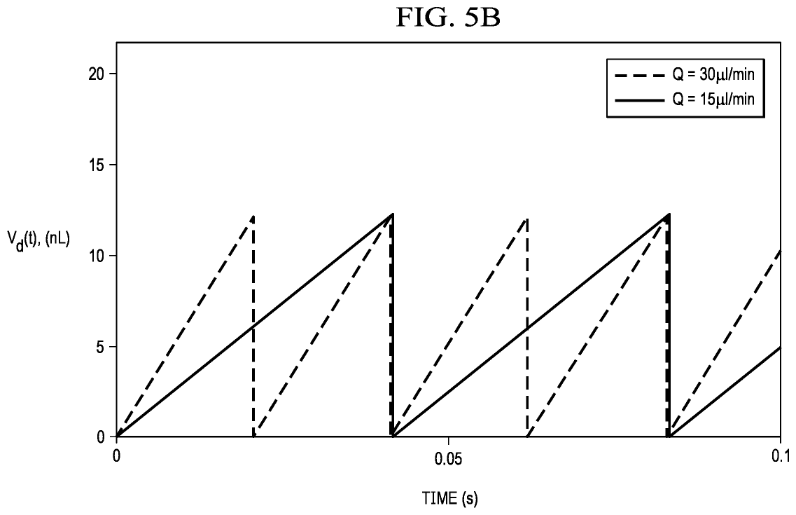

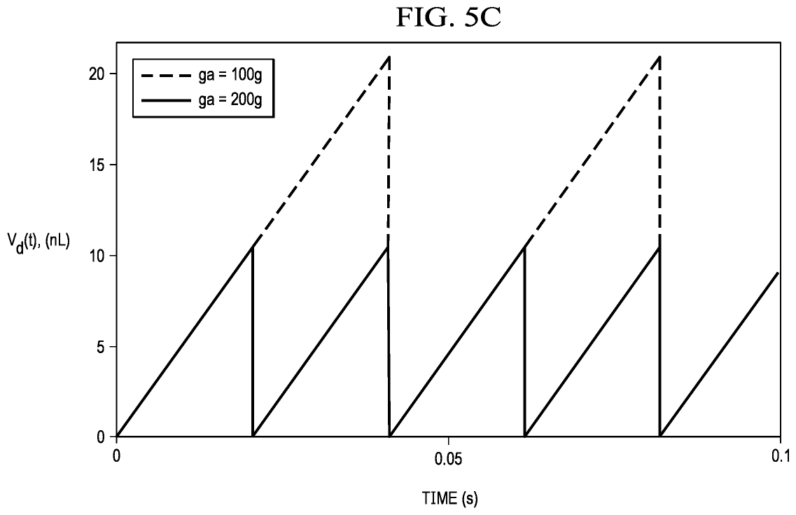

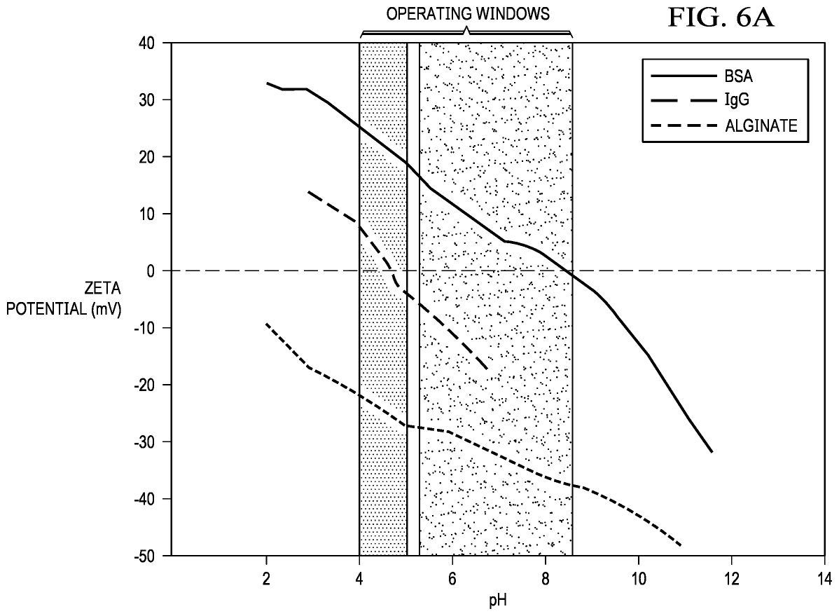

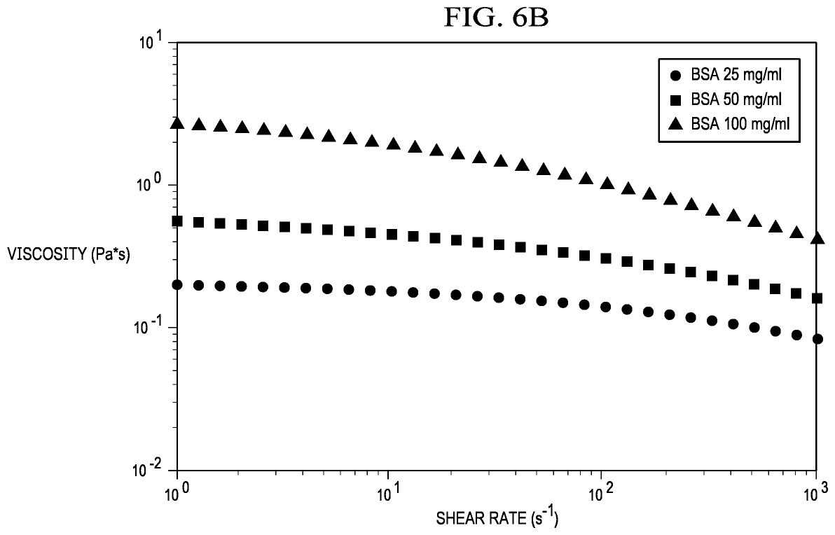

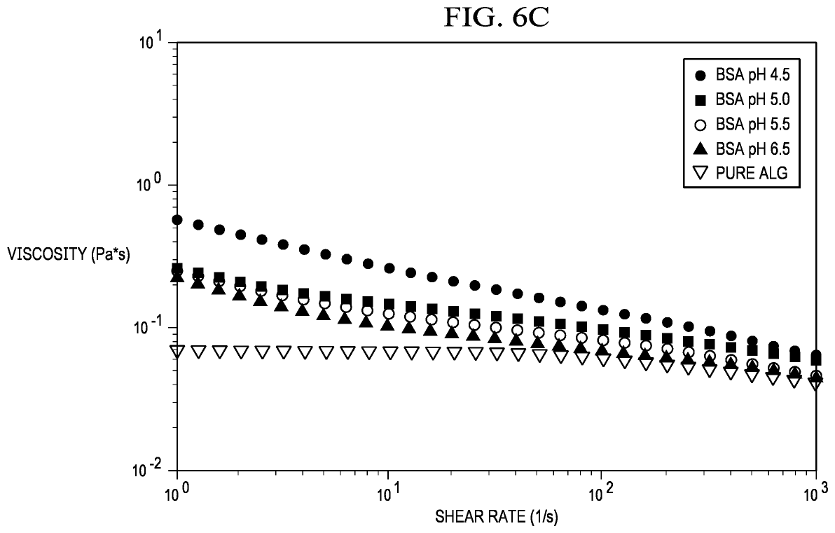

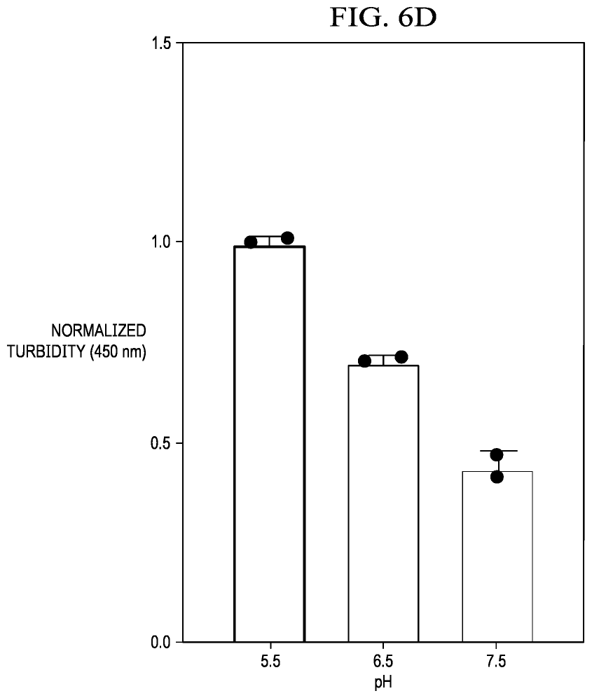

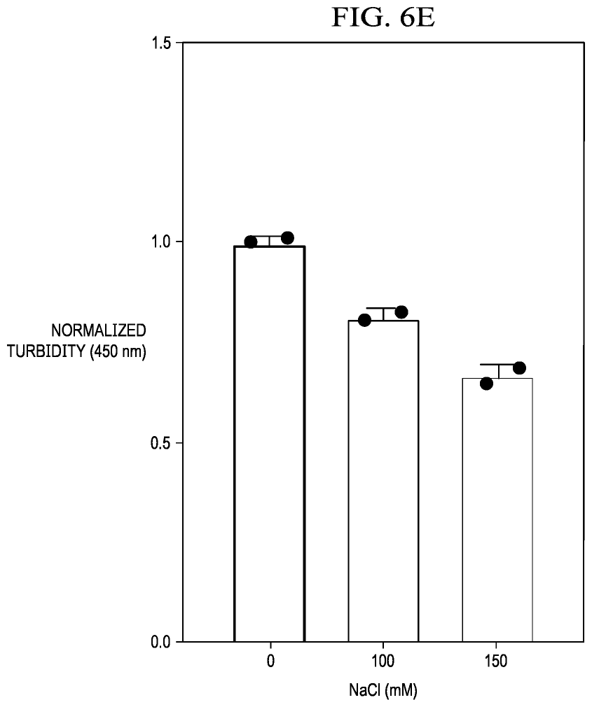

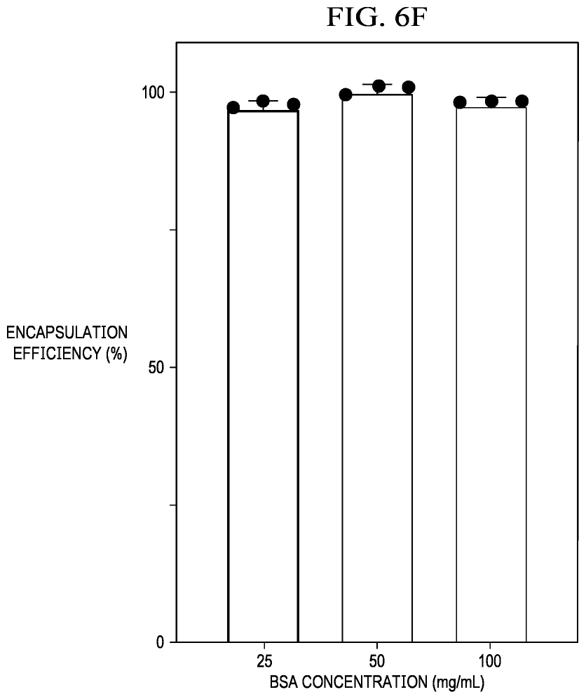

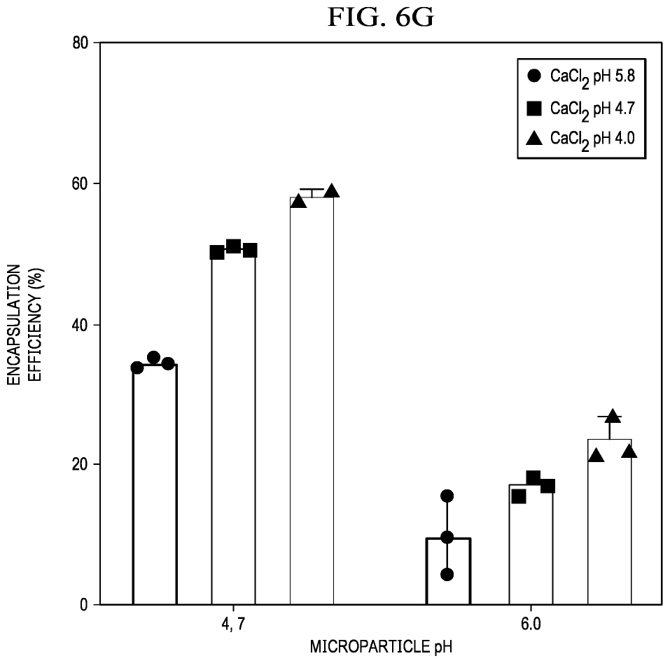

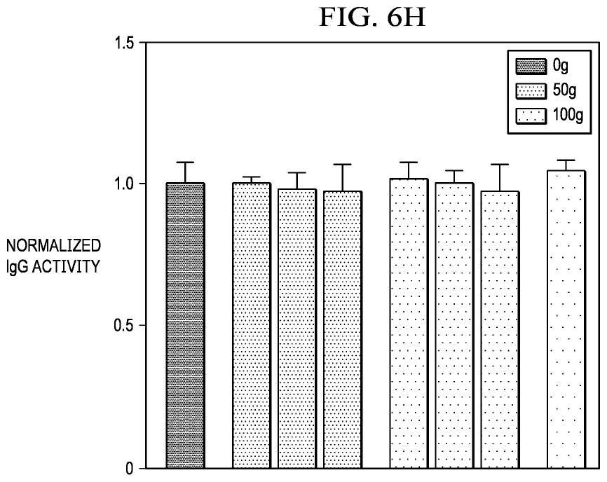

The droplet will detach at the specific time for which V(t_{d})=V_{d}, V=πdσ/ρg_{eq}. For a constant Q and gₐ, the ejection is periodic, so V_{d}=Q·τₑⱼ with τₑⱼ being the droplet ejection period. By increasing Q, the droplet detachment period would decrease, and vice versa (FIG. 5B). Please note that in all these scenarios (varying Q), the droplet volume V_{d} at detachment would stay the same. Only by changing gₐ the droplet size would change (FIG. 5C). It is convenient to refer to this mode as Constant Mode (CM).

Although the present disclosure has been described with reference to certain embodiments thereof, other embodiments are possible without departing from the present disclosure. The spirit and scope of the appended aspects should not be limited, therefore, to the description of the preferred embodiments contained herein. All embodiments that come within the meaning of the aspects, either literally or by equivalence, are intended to be embraced therein.

Furthermore, the advantages described above are not necessarily the only advantages of the disclosure, and it is not necessarily expected that all of the described advantages will be achieved with every embodiment of the disclosure.

1. A printed composition comprising:
a liquid droplet prior to crosslinking and a gelled particle after crosslinking, the liquid droplet comprising a formulation including a hydrogel precursor and a biologic, and the gelled particle comprising a cross-linked hydrogel matrix with the biologic dispersed therein, wherein the formulation has a viscosity in a range from about 100 mPa·s to about 500,000 mPa·s.

2. The printed composition of claim 1, wherein the hydrogel matrix comprises alginate, agar, agarose, carboxymethylcellulose, carrageenan, chitosan, chondroitin sulfate, collagen, dextran, fibrin, gelatin, hyaluronate, hydroxyethylcellulose, xanthan, polylysine, poly(acrylic) acid, poly(ethylene glycol) and its derivatives, cellulose and its derivatives, poly(propylene glycol) and its derivatives, polylactide and its derivatives, poly(glycolic acid) and its derivatives, poly(propylene fumarate) and its derivatives, polycaprolactone and its derivatives, polyhydroxybutyrate and its derivatives, polyacrylates and derivatives, poly(vinylpyrrolidone) and derivatives, and/or poly(ethylenimine) and its derivatives, and
wherein the hydrogel precursor comprises an alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and its derivatives, a precursor for cellulose and its derivatives, a precursor for poly(propylene glycol) and its derivatives, a precursor for polylactide and its derivatives, a precursor for poly(glycolic acid) and its derivatives, a precursor for poly(propylene fumarate) and its derivatives, a precursor for polycaprolactone and its derivatives, a precursor for polyhydroxybutyrate and its derivatives, a precursor for polyacrylates and derivatives, a precursor for poly(vinylpyrrolidone) and derivatives, and/or a precursor for poly(ethylenimine) and its derivatives.

3. The printed composition of claim 1, wherein the biologic comprises a protein, hormone, peptide, nucleic acid, mammalian cell, micro-organism, small molecule, bacteria, drug (e.g., an antibody-based drug, such as monoclonal antibodies, antibody-drug conjugates, bispecific antibodies), cytokine (e.g., interleukin, interferon, tumor necrosis factor, chemokine, transforming growth factor beta, growth factor), insulin, Botulinum toxin type A, Botulinum toxin type B, bovine serum albumin (BSA), human immunoglobulin G (IgG), Fc fusion protein, anticoagulant, blood factor, bone morphogenetic protein, engineered protein scaffold, enzyme, and/or thrombolytic.

4. The printed composition of claim 1, wherein the biologic is homogeneously dispersed in the cross-linked hydrogel matrix.

5. The printed composition of claim 1, wherein the gelled particle comprises a hydrogel-to-biologic ratio in a range from about 1:1 to about 1:1000.

6. The printed composition of claim 1, further comprising a shell encapsulating the gelled particle, the shell comprising a biocompatible polymer.

7. (canceled)

8. The printed composition of claim 1, wherein the gelled particle comprises an encapsulation efficiency of at least about 55%.

9. The printed composition of claim 1, wherein the gelled particle has a diameter in a range from about 10 microns to about 2 mm.

10. The printed composition of claim 1, wherein the formulation includes the hydrogel precursor at a concentration of at least about 20 mg/mL and/or as high as about 1000 mg/mL.

11. The printed composition of claim 1, wherein the formulation includes the biologic at a concentration of at least about 20 mg/mL and/or as high as about 1000 mg/mL.

12. (canceled)

13. The printed composition of claim 1, wherein the formulation further comprises an excipient to stabilize the biologic, the excipient comprising one or more of the following: a buffering agent, such as citrate, phosphate, acetate and/or histidine buffer; an amino acid, such as L-arginine hydrochloride and/or L-glutamic acid, antioxidant, such as ascorbic acid, methionine, and/or ethylenediaminetetraacetic acid (EDTA); a surfactant, such as Polysorbate 80, Polysorbate 20, Brij 30 and Brij 35 and Pluronic F127; and/or a preservative such as benzyl alcohol, cresol, phenol, and/or chlorobutanol.

14. The printed composition of claim 1, wherein the formulation further includes an adjuvant comprising one or more of the following: an aluminum salt, such as amorphous aluminum hydroxyphosphate sulfate (AAHS), aluminum hydroxide, aluminum phosphate, potassium aluminum sulfate, and/or cytosine phosphoguanine (CpG).

15. The printed composition of claim 1, wherein delivery or administration of the gelled particle into the human body may include one or more of the following: uricular, buccal, conjunctival, cutaneous, dental, electro-osmotical, endocervical, endosinusial, endotracheal, enteral, epidural, extra amniotical, extracorporeal, infiltration, interstitial, intra-abdominal, intra-amniotical, intra-arterial, intra-articular, intrabiliary, intrabronchial, intrabursal, intracardial, intracartilaginous, intracaudal, intracavernous, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastrical, occlusive dressing technique, ophthalmical, oral, oropharyngeal, parenteral, percutaneous, periarticular, peridural, perineural, periodontal, rectal, inhalation, retrobulbar, soft tissue, subarachnoidial, subconjunctival, subcutaneous, sublingual, submucosal, topical, transdermal, transmucosal, transplacental, transtracheal, transtympanic, ureteral, urethral, and/or vaginal administration.

16. A method of acoustophoretically printing a composition, the method comprising:
arranging a nozzle within a first fluid, the nozzle having a nozzle opening;
generating an acoustic field in the first fluid by an oscillating emitter;
driving a formulation comprising a hydrogel precursor and a biologic out of the nozzle so as to form a pendant droplet comprising the formulation at the nozzle opening;
detaching the pendant droplet by acoustic forces from the acoustic field, the formulation thereby being released in the first fluid as a liquid droplet; and
crosslinking the liquid droplet to form a gelled particle comprising a crosslinked hydrogel matrix with the biologic dispersed therein.

17. The method of claim 16, wherein the crosslinking is initiated by a crosslinking reagent, heat/temperature, irradiation, and/or a change in pH.

18. The method of claim 16, wherein the liquid droplet is deposited in a bath, and wherein the crosslinking takes place in the bath.

19. The method of claim 16, wherein the liquid droplet is deposited on a substrate, and wherein the crosslinking takes place on or prior to reaching the substrate.

20. The method of claim 16, wherein the formulation includes the hydrogel precursor at a concentration of at least about 20 mg/mL and/or as high as about 1000 mg/mL, and/or wherein the hydrogel precursor comprises an alginate precursor, an agar precursor, an agarose precursor, a carboxymethylcellulose precursor, a carrageenan precursor, a chitosan precursor, a chondroitin sulfate precursor, a collagen precursor, a dextran precursor, a fibrin precursor, a gelatin precursor, a hydroxyethylcellulose precursor, a hyaluronate precursor, a xanthan precursor, a polylysine precursor, a poly(acrylic) acid precursor, a precursor for poly(ethylene glycol) and its derivatives, a precursor for cellulose and its derivatives, a precursor for poly(propylene glycol) and its derivatives, a precursor for polylactide and its derivatives, a precursor for poly(glycolic acid) and its derivatives, a precursor for poly(propylene fumarate) and its derivatives, a precursor for polycaprolactone and its derivatives, a precursor for polyhydroxybutyrate and its derivatives, a precursor for polyacrylates and derivatives, a precursor for poly(vinylpyrrolidone) and derivatives, and/or a precursor for poly(ethylenimine) and its derivatives.

21. The method of claim 16, wherein the formulation includes the biologic at a concentration of at least about 20 mg/mL and/or as high as about 1000 mg/mL, and/or
wherein the biologic comprises a protein, hormone, peptide, nucleic acid, mammalian cell, micro-organism, small molecule, bacteria, drug (e.g., an antibody-based drug, such as monoclonal antibodies, antibody-drug conjugates, bispecific antibodies), cytokine (e.g., interleukin, interferon, tumor necrosis factor, chemokine, transforming growth factor beta, growth factor), insulin, Botulinum toxin type A, Botulinum toxin type B, bovine serum albumin (BSA), human immunoglobulin G (IgG), monoclonal antibody (mAb), Fc fusion protein, anticoagulant, blood factor, bone morphogenetic protein, engineered protein scaffold, enzyme, and/or thrombolytic.

22. (canceled)

23. A printed composition comprising:
a gelled particle comprising a cross-linked hydrogel matrix with a biologic dispersed therein,
wherein the gelled particle is obtained by crosslinking a liquid droplet comprising a formulation including a hydrogel precursor and the biologic, the formulation having a viscosity in a range from about 100 mPa·s to about 500,000 mPa·s.
